(19) United States
(12) Patent Application Publication
(10) Pub. No.: US 2018/0110954 A1
(43) Pub. Date: Apr. 26, 2018

(54) VENTILATOR APPARAUS AND SYSTEMS

(71) Applicant: SMITHS MEDICAL INTERNATIONAL LIMITED, Ashford (GB)

(72) Inventors: Anthony Lucio Belisario, Luton (GB); Paul James Leslie Bennett, Marston Moretaine (GB); Robert James Burchell, Baldock (GB); Mohammad Qassim Mohammad Khasawneh, Milton Keynes (GB); Mark Charles Oliver, St Albans (GB)

(73) Assignee: SMITHS MEDICAL INTERNATIONAL LIMITED, Ashford (GB)

(21) Appl. No.: 15/567,669

(22) PCT Filed: Apr. 14, 2016
(86) PCT No.: PCT/GB2016/000081

§ 371 Date: Oct. 19, 2017

(30) Foreign Application Priority Data
Apr. 29, 2015 (GB) 1507549.2

(51) Int. Cl. A61M 16/10; A61M 16/00; A61M 16/20; B01D 53/053; B01D 53/04

(52) U.S. Cl. CPC A61M 16/101; A61M 16/0063; A61M 16/208; B01D 53/053; B01D 53/0446; A61M 16/105; A61M 2205/75; B01D 2253/108; B01D 2256/12; B01D 2257/102; B01D 2259/402; B01D 2259/4533; B01D 2259/4541; A61M 2202/0208

(57) ABSTRACT

Ventilator apparatus includes a pump (41) connected to supply pressurised air both to an air reservoir (23) and to an oxygen concentrator (70) that supplies pressurised oxygen to an oxygen reservoir (24). The outlet (50) of the air reservoir (23) is connected to the inlet of a breathing circuit (30) via an entrainment device (56) so that pressurised air from the reservoir entrains atmospheric air. The outlet (84) of the oxygen reservoir (24) is connected via oxygen tubing (99) to the patient end of the breathing circuit (30). A patient valve (90) at the patient end (93) of the breathing circuit (30) opens to allow the patient to exhale via openings (97) in the valve. The oxygen supply is switched to supply oxygen to the breathing circuit (30) during the expiratory phase so that oxygen in the circuit is inhaled during a subsequent inhalation phase.

This invention relates to ventilator apparatus of the kind including a source of compressed air from the atmosphere and a reservoir of compressed air.

Portable gas-powered, pneumatic ventilators are widely used in both emergency and transport situations. The ventilators can be rugged and simple to operate, which makes them especially suitable for use outside hospitals and by less qualified people, such as paramedics. Such ventilators are usually powered by compressed oxygen, from a cylinder via a reducing valve and most have the ability to entrain air allowing the delivery of between 100% and 50% oxygen. In remote or disaster circumstances, however, the availability of compressed oxygen may be very limited. Pneumatic ventilators, although designed to be powered by compressed oxygen at between 4 and 6 bars, can also be driven by compressed air with certain associated variation in their calibration but do not enable the oxygen concentration of the gas supplied to the patient to be increased. In many circumstances, such as emergency situations in remote areas, compressed air and oxygen may not be available or is only available in restricted quantities. If electrical power is available, such as from a mains supply or a vehicle battery, it is possible to use electrically-powered ventilators to ventilate or resuscitate. Such apparatus includes a pump or electrical compressor that supplies compressed air to a pressure vessel. The outlet of the pressure vessel is supplied, in a regulated manner to the patient. Ventilators driven in this way can only deliver oxygen to the patient at the same concentration as in air, that is, at 21%.

It is an object of the present invention to provide alternative ventilator apparatus and systems.

According to one aspect of the present invention there is provided ventilator apparatus of the above-specified kind, characterised in that the apparatus also includes an oxygen concentrator including a reservoir of compressed oxygen, a gas circuit connecting the outlet of the source of compressed air to both the reservoir of compressed air and to the oxygen concentrator so that air is supplied to both the air reservoir and the oxygen concentrator from a common source, such that both air and oxygen can be supplied to a patient breathing circuit connected with the ventilator apparatus.

The source of compressed air preferably includes an air pump. The apparatus may be arranged to supply oxygen from the reservoir of compressed oxygen to the patient breathing circuit at times when the air from the air reservoir is not being supplied to the patient breathing circuit. The oxygen concentrator preferably includes two molecular sieves connected in parallel and operated to discharge oxygen to the oxygen reservoir alternately. The air reservoir, oxygen reservoir and molecular sieves are preferably of cylindrical shape arranged vertically of the apparatus below an upper unit, and that the upper unit includes user interface controls and a connector for a breathing circuit. The apparatus preferably includes an air entrainment device connected with an outlet of the reservoir of compressed air such that air supplied to the patient is a mixture of air from the air reservoir and air entrained from atmosphere. The air entrainment device may include an oxygen inlet, the apparatus including an oxygen supply path extending between the oxygen inlet and an inlet of the oxygen reservoir.

According to another aspect of the present invention there is provided a ventilator system including ventilator apparatus according to the above one aspect of the present invention and a patient breathing circuit including a breathing tube connected at one end to receive air from the air reservoir and an oxygen tube connected at one end to receive oxygen from the oxygen reservoir and opening at its opposite end in the region of the opposite end of the breathing circuit.

According to a further aspect of the present invention there is provided a ventilator system including a breathing circuit and a ventilator apparatus including a source of compressed air from the atmosphere and a reservoir of compressed air, characterised in that the apparatus also includes an oxygen concentrator including a reservoir of compressed oxygen, a gas circuit connecting the outlet of the source of compressed air to both the reservoir of compressed air and to the oxygen concentrator so that air is supplied to both the air reservoir and the oxygen concentrator from a common source, and that the breathing circuit is connected with the ventilator apparatus such that both air and oxygen are supplied to a patient breathing circuit.

The breathing circuit preferably includes a patient valve with a valve element arranged to open an outlet to atmosphere when the patient exhales, and that the opposite end of the oxygen tube connects with the interior of the patient valve.

A ventilator system and apparatus, both according to the present invention, will now be described, by way of example, with reference to the accompanying drawings, in which:

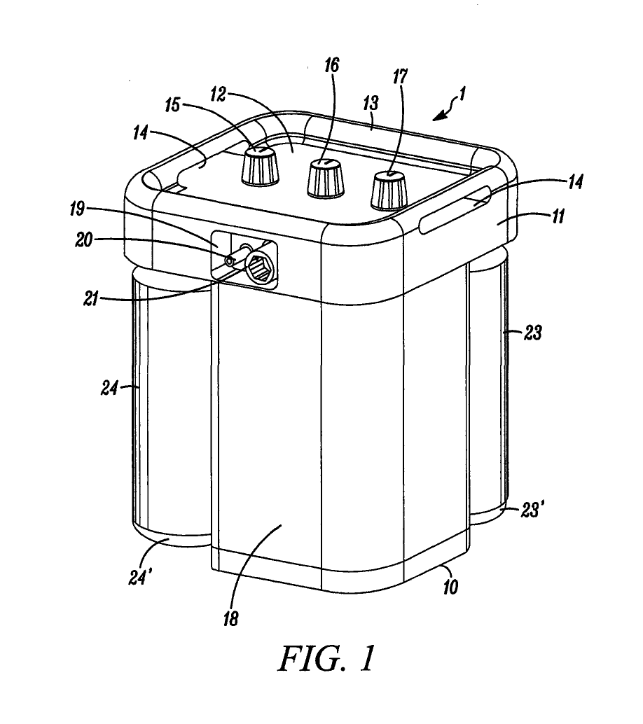

FIG. 1 is a perspective view of the apparatus;

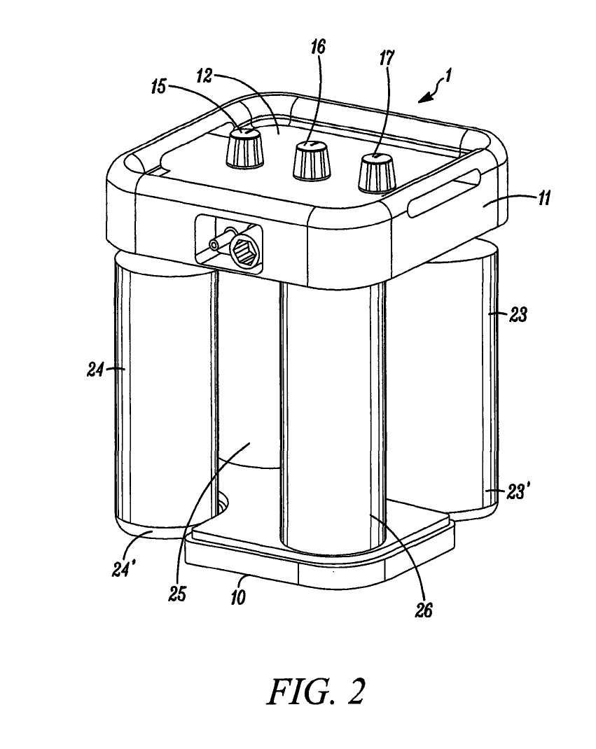

FIG. 2 is a perspective view of the apparatus with a front panel removed to show the interior;

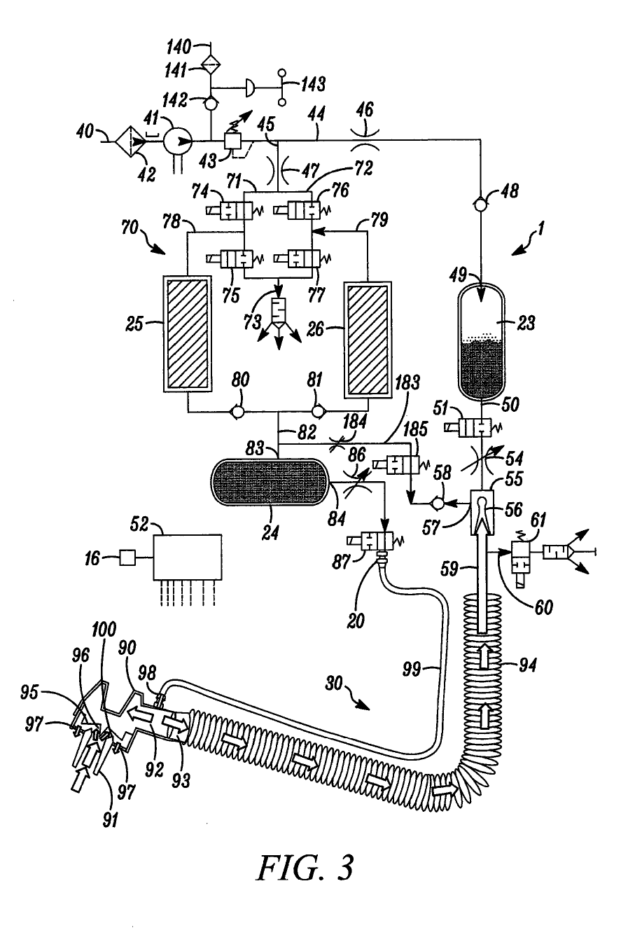

FIG. 3 is a circuit diagram of the system during an expiratory phase; and

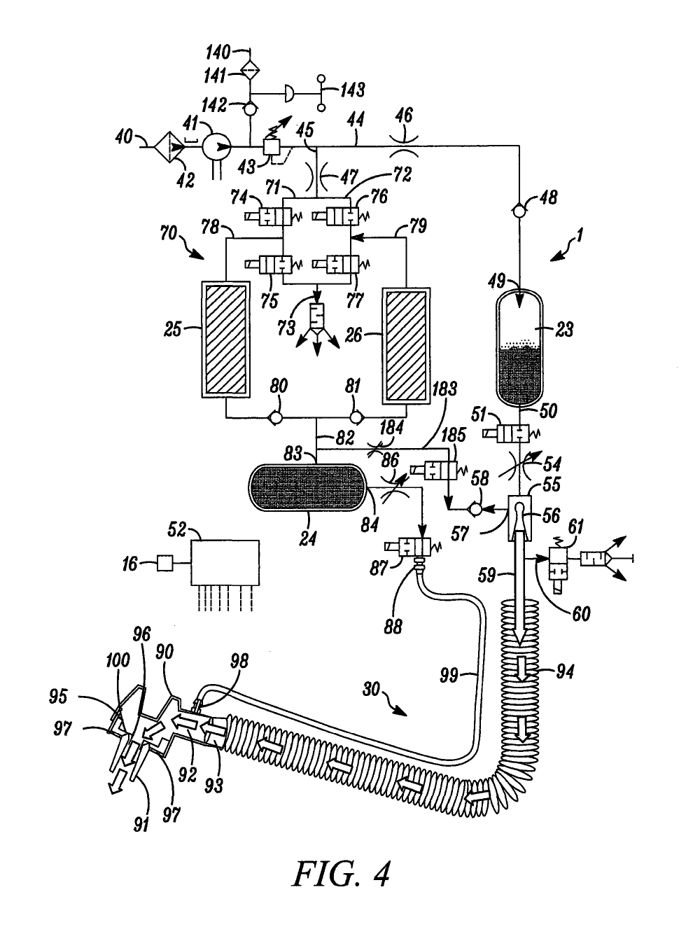

FIG. 4 shows the same circuit as in FIG. 3 but during an inspiratory phase.

With reference first to FIGS. 1 and 2, which show the ventilator apparatus 1 without any associated breathing circuit, the apparatus can be seen to have a generally rectangular section when viewed from above, a flat base 10 for standing on the floor or a table or the like and an upper unit 11 with a horizontal upper surface 12 surrounded by a shallow wall 13 formed with openings 14 at opposite sides so that the apparatus can be gripped and carried by using a handle or straps (not shown) fitted through the openings. The openings 14 also allow any pooled liquid, such as rainwater, to drain away from the upper surface 12. The upper surface 12 provides a user interface supporting three control knobs, namely an oxygen flow control knob 15, a ventilator frequency control knob 16 and a ventilator flow control knob 17. Towards the upper end of the front wall 18 there is a rectangular recess 19 within which project an oxygen connector 20 and a breathing circuit connector 21.

The apparatus 1 includes two reservoirs, namely an air reservoir 23 and an oxygen reservoir 24 both being of cylindrical shape and standing vertically at opposite corners of the apparatus. The lower end 23′ and 24′ of the reservoirs 23 and 24 are closed and openings at their upper ends (not visible) are connected in the ventilator circuit as will be described later. The reservoirs 23 and 24 could be low-cost, blow moulded disposable PET bottles of the kind used to contain drinks since these could simply be replaced periodically to avoid the need for cleaning. Also visible in FIG. 1, at the other opposite corners of the apparatus, are two molecular sieves 25 and 26 in the form of vertically-oriented cylinders containing zeolite, of the kind conventionally used in oxygen concentrators. Electrical connection is made to the apparatus by an electrical socket (not shown) on the rear wall of the apparatus, opposite the front wall 18. The electrical connection could be of mains voltage or of a lower voltage provided by vehicle batteries. The apparatus 1 includes other components not visible in FIGS. 1 and 2, which will be described with reference to FIGS. 3 and 4.

Turning first to FIG. 3, this shows the circuit of the ventilator apparatus 1 schematically and also shows the breathing circuit 30 by which gas is supplied to and from the patient. FIG. 3 shows the system during an expiratory phase of the ventilation cycle. The apparatus 1 has an inlet 40 for atmospheric air, which is connected to an air compressor or pump 41 via a filter 42, which provides a source of compressed air. The outlet of the compressor 41 connects to a gas circuit including a pressure regulator and water trap 43, which regulates the pressure to approximately 2 bar. The outlet of the regulator 43 is split along two paths namely an air supply path 44 and an oxygen supply path 45. The proportion of air that flows along these two paths 44 and 45 is balanced for the particular demand of the apparatus by respective restrictors 46 and 47 in each path.

The apparatus may optionally include an additional, or alternative, source of compressed air in the form of an additional air inlet 140 adapted to be connected to an external source of compressed medical air at around 4-6 bar. This enables the apparatus to be powered instead from an air compressor in a medical facility or vehicle. The inlet 140 connects to the gas circuit upstream of the regulator 43 via a filter 141 and non-return valve 142. The outlet of the filter 141 also connects with a pressure switch 143 that is arranged to disconnect power to the pump 41 when compressed air is connected at the inlet 140. The additional air inlet 140 could include an air connector mounted in a recess (not shown) on the rear face of the apparatus 1.

The air supply path 44 extends via a one-way/non-return valve 48 to the inlet 49 of the air reservoir 23. Although FIGS. 3 and 4 show the inlet 49 and outlet 50 of the reservoir 23 as being at opposite ends, this is only schematic and, in the present example both the inlet and outlet are at the same end. The outlet 50 of the air reservoir 23 is connected to a solenoid valve 51, which has a normally-closed state, as shown, but which can be changed to an open state by electrical signals from a control unit 52. The control unit 52 receives an output from the ventilator frequency control knob 16 to adjust the frequency of opening the solenoid valve 51 and other solenoid valves to be described later. The closed solenoid valve 51 prevents air escaping from the reservoir 23 and thereby allows the compressor 41 to build up pressure in the reservoir. The outlet of the solenoid valve 51 is connected via a variable restrictor 54, which is adjusted by the ventilator flow control knob 17. The outlet of the restrictor 54 connects to the jet inlet 55 of an air entrainment device 56. The air entrainment device 56 has a second inlet 57 open to atmosphere via a one-way/non-return valve 58, so that, when air flows through the jet inlet 55 it entrains air from atmosphere via inlet 57. During the expiratory phase there is no flow through the gas jet inlet 55 and no air entrainment, with the valve 58 being closed. The outlet of the air entrainment device 56 connects with the breathing circuit 30 via a T-piece connector 59 the side arm 60 of which connects with a second solenoid valve 61. The solenoid valve 61 is normally open so that air can vent to atmosphere, the valve being controlled by signals from the control unit 52 to close during inspiration.

The oxygen supply path 45 connects with an oxygen concentrator 70 after the restrictor 47. The supply path 45 divides into two parallel paths, namely a left-hand path 71 and a right-hand path 72. Both paths 71 and 72 connect with an atmospheric vent 73 via a respective series arrangement of two solenoid valves, namely a left-hand series of two solenoid valves 74 and 75 and a right-hand series of two solenoid valves 76 and 77. The junction between the two solenoid valves in each pair is connected by respective air lines 78 and 79 to the inlet of respective molecular sieves 25 and 26. The outlets of the sieves 25 and 26 are connected via respective one-way/non-return valves 80 and 81 to a common outlet line 82 extending to the inlet 83 of the oxygen reservoir 24. The inlet 83 and outlet 84 are shown schematically in FIGS. 3 and 4 as being at different locations but, in practice, in the present example are both at the opening of the bottle that provides the oxygen reservoir 24. The outlet 84 of the oxygen reservoir 24 extends to the oxygen outlet connector 20 via a variable restrictor 86, controlled by the oxygen flow control knob 15, and a solenoid valve 87 that is normally open during the expiratory phase but is closed by signals from the control unit 52 during the inspiratory phase. Initially, therefore, valves 74 and 77 are open and valves 75 and 76 are held closed (as shown in FIG. 3) so that air is supplied under pressure along the paths 71 and 78 to the inlet of the left-hand molecular sieve 25. The oxygen in the air flows readily through the zeolite material in the sieve 25 but the nitrogen in the air cannot pass through the zeolite so it collects at the upper end of the sieve. The oxygen flows from the lower end of the sieve 25 through the non-return valve 80 but is blocked from flowing through the non-return valve 81 so it flows instead into the oxygen reservoir 24. At the same time as the left-hand sieve 25 is charging the oxygen reservoir 24, the upper end of the right-hand sieve 26 is open to atmosphere via the open solenoid valve 77, thereby discharging nitrogen that has built up in that sieve from a preceding cycle. Oxygen that builds up in the oxygen reservoir 24 can only flow out to the patient when the solenoid valve 87 is open, that is, during the expiratory phase. The solenoid valve 87 also assumes its normally open state allowing oxygen to flow to the breathing circuit 30 when cyclical ventilation is not required, thereby enabling oxygen supplementation to be given to patients who are breathing spontaneously. The gas circuit is shown as also including an optional oxygen supply path 183 between the inlet 83 of the oxygen reservoir 24 and the air entrainment inlet 57 of the entrainment device 56. The supply path 183 includes a restrictor 184 and a normally-dosed solenoid valve 185, which is opened by signals form the control unit 52 during the inspiratory phase (FIG. 4). The effect of this is to deliver a small bleed of oxygen into the entrained air stream during inhalation to increase further the delivered oxygen concentration.

Where only oxygen is required, without cyclical ventilation, a conventional oxygen therapy mask or cannula circuit (not shown) could be attached to the oxygen outlet connector 20.

The breathing circuit 30 includes a patient valve 90 with a patient outlet 91 connected to a patient interface, such as a face mask or the like (not shown). The valve 90 has an inlet 92 connected to the outlet end 93 of a flexible, corrugated breathing tube 94. The patient valve 90 includes a flexible valve element 95 with a central duck-bill formation 96. The housing of the valve 90 has several outlet openings 97 around the patient outlet 91. The inlet 92 of the patient valve 90 has a small diameter oxygen inlet 98 close to its end. The circuit 30 includes a small bore oxygen tube 99 extending from the oxygen outlet connector 20 along the side of the breathing tube 94 to the oxygen inlet 98 on the patient valve 90.

During the expiratory phase, the flexible valve element 95 is lifted by pressure from the patient to enable the patient to exhale via the outlet openings 97. The solenoid valve 51 connected at the outlet of the air reservoir 23 is closed, thereby preventing air flowing out of the reservoir to the entrainment device 56 and to the machine end of the breathing tube 94, which is open to atmosphere via the solenoid valve 61. Oxygen from the oxygen reservoir 24 flows via the open solenoid valve 87 and the connector 20 to the patient end of the breathing circuit 30 via the small bore oxygen tubing 99 and the oxygen inlet 98. As the duckbill formation 96 on the patient valve element 95 is closed by the expiration pressure from the patient, oxygen flows rearwardly along the breathing tube 94 towards its machine end. The open solenoid valve 61 connected at the machine end of the breathing tube 94 enables residual air and air mixtures in the breathing tube to be flushed out of the tube so that it fills initially with relatively pure oxygen.

Operation of the ventilator system during the inspiratory phase will now be described with reference to FIG. 4. During this phase the control unit 52 drives the solenoid valve 87 at the outlet of the oxygen reservoir 24 to close so that oxygen cannot flow to the breathing circuit 30, thereby enabling the oxygen in the oxygen reservoir to be recharged. The solenoid valve 51 at the outlet of the air reservoir 23, however, is driven to open so that air can flow out of the air reservoir to the jet inlet 55 of the air entrainment device 56. This draws atmospheric air from the inlet 57 to mix with the pressurised air supplied to the jet inlet 55. This air mixture flows into the machine end of the breathing tube 94 since the control unit 52 drives the venting solenoid valve 61 to the closed position preventing air escaping to atmosphere. The air supplied to the machine end of the breathing tube 94 mixes with the oxygen supplied to the tube during the previous phase so that an oxygen-enriched air mixture flows along the breathing tube to the patient valve 90. The pressure this generates in the patient valve 90 forces the valve element 95 against a sealing lip 100 around the patient outlet 91, thereby preventing gas escaping via the openings 97. Instead, the pressure in the patient valve 90 forces the duck-bill formation 96 open so that the air and oxygen mixture can flow to the patient. The compressor 41 continues to supply air to both the air reservoir 23 and one or other of the molecular sieves 25 or 26 during both inspiratory and expiratory phases.

The arrangement of the present invention uses a common compressor or pump 41 to drive both the ventilator air supply and the oxygen concentrator. This enables the apparatus to be compact and keeps its weight and cost to a minimum. The apparatus can be used readily to provide an air and oxygen mixture, as described above, or cycled air ventilation only, or a continuous supply of oxygen without air.

1-10. (canceled)

11. Ventilator apparatus including a source of compressed air from the atmosphere and a reservoir of compressed air, characterised in that the apparatus also includes an oxygen concentrator including a reservoir of compressed oxygen, a gas circuit connecting the outlet of the source of compressed air to both the reservoir of compressed air and to the oxygen concentrator so that air is supplied to both the air reservoir and the oxygen concentrator from a common source such that both air and oxygen can be supplied to a patient breathing circuit connected with the ventilator apparatus.

12. Apparatus according to claim 11, characterised in that the source of compressed air includes an air pump.

13. Apparatus according to claim 11, characterised in that the apparatus is arranged to supply oxygen from the reservoir of compressed oxygen to the patient breathing circuit at times when the air from the air reservoir is not being supplied to the patient breathing circuit.

14. Apparatus according to claim 11, characterised in that the oxygen concentrator includes two molecular sieves connected in parallel and operated to discharge oxygen to the oxygen reservoir alternately.

15. Apparatus according to claim 14, characterised in that the air reservoir, oxygen reservoir and molecular sieves are of cylindrical shape arranged vertically of the apparatus below an upper unit, and that the upper unit includes user interface controls and a connector for a breathing circuit.

16. Apparatus according to claim 11, characterised in that the apparatus includes an air entrainment device connected with an outlet of the reservoir of compressed air such that air supplied to the patient is a mixture of air from the air reservoir and air entrained from atmosphere.

17. Apparatus according to claim 16, characterised in that the air entrainment device includes an oxygen inlet and that the apparatus includes an oxygen supply path extending between the oxygen inlet and an inlet of the oxygen reservoir.

18. A ventilator system including ventilator apparatus comprising:
a source of compressed air from the atmosphere and a reservoir of compressed air, an oxygen concentrator including a reservoir of compressed oxygen, a gas circuit connecting the outlet of the source of compressed air to both the reservoir of compressed air and to the oxygen concentrator so that air is supplied to both the air reservoir and the oxygen concentrator from a common source such that both air and oxygen can be supplied to a patient breathing circuit connected with the ventilator apparatus; and
a patient breathing circuit including a breathing tube connected at one end to receive air from the air reservoir and an oxygen tube connected at one end to receive oxygen from the oxygen reservoir and opening at its opposite end in the region of the opposite end of the breathing circuit.

19. A ventilator system including a breathing circuit and a ventilator apparatus including a source of compressed air from the atmosphere and a reservoir of compressed air, characterised in that the apparatus also includes an oxygen concentrator including a reservoir of compressed oxygen, a gas circuit connecting the outlet of the source of compressed air to both the reservoir of compressed air and to the oxygen concentrator so that air is supplied to both the air reservoir and the oxygen concentrator from a common source, and that the breathing circuit is connected with the ventilator apparatus such that both air and oxygen are supplied to a patient breathing circuit.

20. A ventilator system according to claim 18, characterised in that the breathing circuit includes a patient valve with a valve element arranged to open an outlet to atmosphere when the patient exhales, and that the opposite end of the oxygen tube connects with the interior of the patient valve.

21. A ventilator system according to claim 19, characterised in that the breathing circuit includes a patient valve with a valve element arranged to open an outlet to atmosphere when the patient exhales, and that the opposite end of the oxygen tube connects with the interior of the patient valve.
